(19) United States
(12) Patent Application Publication
(10) Pub. No.: US 2023/0240985 A1
(43) Pub. Date: Aug. 3, 2023

(54) STABLE PHARMACEUTICAL COMPOSITION OF TOPIRAMATE

(71) Applicant: ALKEM LABORATORIES LIMITED, Mumbai (IN)

(72) Inventors: Arun Kumar Pandey, Navi Mumbai (IN); Umadoss Pothuvan, Navi Mumbai (IN); Gaurav Chaurasia, Kalyan-Dombivali (East) (IN); Chaitanya Shrisath, Kalyan-Dombivali (East) (IN); Ulhas Dhuppad, Nashik (IN); Abhay Rahangdale, Navi Mumbai (IN)

(21) Appl. No.: 18/160,866

(22) Filed: Jan. 27, 2023

(30) Foreign Application Priority Data
Jan. 28, 2022 (IN) IN 202221004749

(51) Int. Cl. A61K 9/00; A61K 9/08; A61K 31/357; A61K 47/10; A61K 47/26; A61K 47/46

(52) U.S. Cl. CPC A61K 9/0053; A61K 9/08; A61K 31/357; A61K 47/10; A61K 47/26; A61K 47/46

(57) ABSTRACT

The present invention relates to a stable pharmaceutical composition of Topiramate and process for the preparation thereof. This invention more particularly relates to a stable aqueous pharmaceutical composition comprising Topiramate and process for the preparation thereof.

### FIELD OF THE INVENTION

The present invention relates to a stable pharmaceutical composition of Topiramate and process for the preparation thereof. This invention more particularly relates to a stable aqueous pharmaceutical composition comprising Topiramate, and process for the preparation thereof.

### BACKGROUND OF THE INVENTION

Topiramate is 2,3:4,5-Di-O-isopropylidine-β-D-fructopyranose sulfamate, and has the following chemical structure:

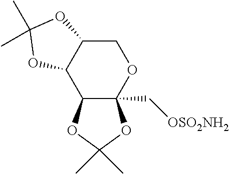

Topiramate is a white crystalline powder with a bitter taste. Topiramate is a sulfamate-substituted monosaccharide. Topiramate is most soluble in alkaline solutions containing sodium hydroxide or sodium phosphate and having a pH of 9 to 10. It is freely soluble in acetone, chloroform, dimethylsulfoxide, and ethanol. The solubility in water is 9.8 mg/mL. Its saturated solution has a pH of 6.3. However, topiramate is not stable in aqueous solution, and therefore has been heretofore formulated primarily in solid dosage forms such as capsules and tablets from which water can easily be excluded.

Topiramate formulations are commercially available in the United States of America under the brand name of Topamax® Capsule, 15 & 25MG; Topamax® Tablet, 25, 50, 100 & 100MG; Qudexy® XR Capsules, 25, 50, 100, 150 & 200MG; Trokendi XR® Capsules, 25, 50, 100 & 200MG and Eprontia™ Oral solution 25MG/ML and are indicated for treatment of Epilepsy: Initial monotherapy for the treatment of partial-onset or primary generalized tonic-clonic seizures in patients 2 years of age and older; adjunctive therapy for the treatment of partial-onset seizures, primary generalized tonic-clonic seizures, or seizures associated with Lennox-Gastaut syndrome in patients 2 years of age and older and Preventive treatment of migraine in patients 12 years of age and older.

PCT application No. WO 2008070670, describes a novel enhanced immediate release formulation of topiramate for oral administration to a mammalian subject, wherein at least 80% of the active compound is dissolved in a time period of not more than 30 minutes. The formulation comprises topiramate as an active ingredient and at least one complexing agent. The application describes a highly soluble complex of topiramate with a cyclodextrin which is selected from a group consisting of hydroxypropyl-β-cyclodextrin, β-cyclodextrin, gamma. -cyclodextrin, and α-cyclodextrin, or its derivative.

PCT application No. WO 2008061226, describes more soluble and bioavailable form of topiramate for once-daily sustained-release dosage form of topiramate or salts thereof wherein the formulation comprises an enhanced immediate release coated bead population in addition to two extended release (XR) coated bead populations, wherein each XR component comprises a release controlling coating which is specific for every population of beads and determines the rate of release of topiramate from the given bead population.

US20140348931 discloses a sustained release formulation of topiramate comprising either two extended release populations, a first extended release population (XR1) and a second extended release population (XR2) or a combination of one immediate release population (IR1) and other extended release population wherein topiramate is present in both the populations and at least one of the populations is present in a matrix form. As per the disclosure made, IR1 population is generally an Immediate Release matrix tablet, XR1 population is matrix tablet or pellets prepared by extrusion/spherization method and XR2 population is always an extended release pellet loaded with drug solution and then coated with extended release layer. Population of any of these two populations is filled in a Capsule.

PCT Appl. No. WO 2014143380, is directed to extended-release topiramate capsule that includes a capsule shell containing a single population of coated particles; wherein each coated particle includes a core and a coating thereon; wherein each particle core includes a homogeneous mixture comprising topiramate throughout its core; and wherein the coating includes one or more release controlling agent(s).

US20160235776 discloses a sustained release topiramate composition comprising a single population of beads, wherein each bead comprises a) an inert carrier b) an inner layer comprising topiramate, c) an outer layer comprising at least one sustained release material and at least one alkalizer, wherein number of beads sufficient to deliver the desired dose may be for example compressed into a tablet, filled into pouches, encapsulated into a capsule of any desirable size. The specification also discloses the use of a specific amount of alkalizer in the coating.

U.S. Pat. No. 4,513,006 discloses Topiramate and its use for treating epilepsy and glaucoma.

PCT application No. WO2005/048981, discloses a controlled release formulation of topiramate in a liquid dosage form.

PCT application No. WO/2006/125774, describes a liquid paediatric formulation of topiramate that is prepared as a “preconcentrate” which contains a relatively low water content, and which is diluted with water to provide dosages for administration to a paediatric patient.

US 20210169844 A1 discloses liquid pharmaceutical composition of topiramate which is non-aqueous which does not contain water in the composition and neither requires addition of aqueous acid or base.

Despite the above-mentioned prior art, there still exists a need for a pharmaceutical composition of topiramate and process for the preparation thereof which is able to overcome the problems associated with the existing formulation such as solubility, stability and impurity, while the most one difficult being a selection of excipients for the development of a stable pharmaceutical compositions of topiramate for oral solution. The commercially available marketed product, EPRONTIA™ 25 mg/mL, is a non-aqueous composition, which does not contain water in the composition and neither requires addition of aqueous acid or base. It has now been found surprisingly that a stable aqueous pharmaceutical composition of topiramate can be prepared which simultaneously achieves the desired stability characteristics and dissolution profile.

### OBJECT OF THE INVENTION

It is an object of the present invention to provide a stable aqueous pharmaceutical composition comprising topiramate and pharmaceutical acceptable excipients.

It is another object of the present invention to provide a stable aqueous pharmaceutical composition of topiramate comprising:
a) Topiramate, as an active ingredient;
b) Polyethylene glycol as solvent;
c) Co-solvent;
d) purified water;
e) sweetener;
f) flavouring agent; and
g) one or more pharmaceutically acceptable excipient.

It is another object of the present invention to provide a stable aqueous pharmaceutical composition of Topirmate comprising:
a) about 2.50% w/v of Topiramate,
b) about 48% w/v of Polyethylene glycol;
c) about 2.0% w/v of methyl paraben;
d) about 0.22% w/v of propyl paraben;
e) about 1% w/v of sucralose;
f) about 1.90% w/v of purified water;
g) about 1.9% w/v of propylene glycol;
h) about 0.05% w/v of mixed berry flavour; and
i) about 61.94% w/v of glycerin.

It is yet another object of the present invention to provide a stable aqueous pharmaceutical composition of Topiramate comprising:
a) about 2.09% w/w of Topiramate;
b) about 40.17% w/w of Polyethylene glycol;
c) about 1.67% w/w of methyl paraben;
d) about 0.19% w/w of propyl paraben;
e) about 0.84% w/w of sucralose;
f) about 1.59% w/w of purified water;
g) about 1.59% w/w of propylene glycol;
h) about 0.05% w/w of mixed berry flavour; and
i) about 51.83% w/w of glycerin.

It is yet another object of the present invention to provide a stable aqueous pharmaceutical composition of Topiramate comprising:
a) about 25 mg/ml of Topiramate;
b) about 480 mg/ml of Polyethylene glycol;
c) about 20 mg/ml of methyl paraben;
d) about 2.2 mg/ml of propyl paraben;
e) about 10 mg/ml of sucralose;
f) about 19 mg/ml of purified water;
g) about 19 mg/ml of propylene glycol;
h) about 0.45 mg/ml of mixed berry flavour; and
i) about 619.4 mg/ml of glycerin.

It is yet another object of the present invention to provide a stable aqueous pharmaceutical composition of Topiramate comprising:
a) about 15-30 mg/ml of Topiramate;
b) about 340-540 mg/ml of Polyethylene glycol;
c) about 12-25 mg/ml of methyl paraben;
d) about 1-5 mg/ml of propyl paraben;
e) about 4-16 mg/ml of sucralose;
f) about 10-30 mg/ml of purified water;
g) about 10-30 mg/ml of propylene glycol;
h) about 0.1-1 mg/ml of mixed berry flavour; and
i) about 530-670 mg/ml of glycerin.

It is another object of the present invention to provide a process for preparation of a stable aqueous pharmaceutical compositions comprising;
a) Topiramate, as an active ingredient;
b) Polyethylene glycol as solvent;
c) Co-solvent;
d) purified water;
e) Sweetener;
f) Flavouring agent; and
g) one or more pharmaceutically acceptable excipient;
   wherein the process comprises dissolving methyl paraben, propyl paraben and propylene glycol in polyethylene glycol 400 and adding Topiramate, sucralose, purified water, mixed berry flavor to obtain clear solution while and adjusting final volume using Glycerin.

### SUMMARY OF THE INVENTION

The present invention provides a stable aqueous pharmaceutical composition comprising topiramate and process for the preparation thereof. The present invention provides a stable pharmaceutical composition of topiramate for oral administration, wherein said composition contains about 2.5% w/v of topiramate.

In one aspect, the present invention provides a stable aqueous pharmaceutical composition of topiramate is a liquid solution.

In another aspect of the present invention, there is provided a stable aqueous pharmaceutical composition of topiramate comprising:
a) Topiramate, as an active ingredient;
b) Polyethylene glycol as solvent;
c) Co-solvent;
d) purified water;
e) Sweetener;
f) Flavouring agent; and
g) one or more pharmaceutically acceptable excipient.

In yet another aspect of the present invention, there is provided a stable aqueous pharmaceutical composition of Topiramate comprising:
a) about 2.50% w/v of Topiramate;
b) about 48% w/v of Polyethylene glycol;
c) about 2.0% w/v of methyl paraben;
d) about 0.22% w/v of propyl paraben;
e) about 1% w/v of sucralose;
f) about 1.90% w/v of purified water;
g) about 1.9% w/v of propylene glycol;
h) about 0.05% w/v of mixed berry flavour; and
i) about 61.94 w/v of glycerin.

In one aspect of the present invention, there is provided a stable aqueous pharmaceutical composition of Topiramate comprising:
a) about 2.09% w/w of Topiramate;
b) about 40.17% w/w of Polyethylene glycol;
c) about 1.67% w/w of methyl paraben;
d) about 0.19% w/w of propyl paraben;
e) about 0.84% w/w of sucralose;
f) about 1.59% w/w of purified water;
g) about 1.59% w/w of propylene glycol;
h) about 0.05% w/w of mixed berry flavour; and
i) about 51.83% w/w of glycerin.

It is yet another aspect of the present invention to provide a stable aqueous pharmaceutical composition of Topiramate comprising:
a) about 25 mg/ml of Topiramate;
b) about 480 mg/ml of Polyethylene glycol;
c) about 20 mg/ml of methyl paraben;
d) about 2.2 mg/ml of propyl paraben;
e) about 10 mg/ml of sucralose;
f) about 19 mg/ml of purified water;
g) about 19 mg/ml of propylene glycol;
h) about 0.45 mg/ml of mixed berry flavour; and
i) about 619.4 mg/ml of glycerin.

It is yet another aspect of the present invention to provide a stable aqueous pharmaceutical composition of Topiramate comprising:
a) about 15-30 mg/ml of Topiramate;
b) about 340-540 mg/ml of Polyethylene glycol;
c) about 12-25 mg/ml of methyl paraben;
d) about 1-5 mg/ml of propyl paraben;
e) about 4-16 mg/ml of sucralose;
f) about 10-30 mg/ml of purified water;
g) about 10-30 mg/ml of propylene glycol;
h) about 0.1-1 mg/ml of mixed berry flavour; and
i) about 530-670 mg/ml of glycerin.

In another aspect of the present invention, there is provided a process for preparation of a stable aqueous pharmaceutical compositions comprising;
a) Topiramate, as an active ingredient;
b) Polyethylene glycol as solvent;
c) Co-solvent;
d) purified water;
e) Sweetener;
f) Flavouring agent; and
g) one or more pharmaceutically acceptable excipient;
   wherein the process comprises dissolving methyl paraben, propyl paraben and propylene glycol in polyethylene glycol 400 and adding Topiramate, sucralose, purified water, mixed berry flavor to obtain clear solution while and adjusting final volume using Glycerin.

### DETAILED DESCRIPTION OF THE INVENTION

Before the present process and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges also encompassed within the invention, subject to any specifically excluded limit in the stated range. Where the stated range includes one or both of the limits, ranges excluding either both of those included limits are also included in the invention.

Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their availability to the applicant prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “stable” as used herein refers to chemical stability of topiramate in aqueous liquid dosage form wherein there is no change in assay values and/or the total impurity is less than 2%, when the dosage form is kept at 25° C./60% RH and 40° C./75% RH for at least 3 months.

The term “composition” as used herein is intended to encompass a product comprising the specified ingredients (and in the specified amount, if indicated), as well as any product which results, directly or indirectly, from combination of the specified ingredients in the specified amounts. By “pharmaceutically acceptable” it is mean that the diluent, excipient or carrier must be compatible with the other ingredients of the formulation and not deleterious to the recipient thereof.

The term “solvent” as used herein refers to an active agent dissolve in solvent. A solvent is a substance that dissolves a solute, resulting in a solution. A solvent is usually a liquid but can also be a solid, a gas, or a supercritical fluid. Water and alcohol mostly used as solvent in the formulation.

The term “co-solvent,” as used herein, refers to a compound having the ability to increase the solubility of a solute. Co-solvents are added to increase the solvent power of the primary substance in the mixture.

The term “aqueous,” as used herein, refers to the composition which requires addition of water in the composition. The amount of water in the formulations of the present invention can range from about 1% w/v to about 6% w/v; from about 1.2% w/v to about 5% w/v; and about 1.5% w/v to about 4% w/v. More preferably, the amount of water present in the compositions of the present invention is between 1.50% w/v to about 5% w/v.

In an embodiment, the present invention provides that the one or more pharmaceutically acceptable excipient(s) constituting the stable aqueous pharmaceutical compositions of topiramate are solvents or co-solvents, preservative, sweetener and other suitable excipients.

In one embodiment of the present invention, there is provided a stable aqueous pharmaceutical composition of topiramate comprising:
a) Topiramate, as an active ingredient;
b) Polyethylene glycol as solvent;
c) Co-solvent;
d) purified water;
e) Sweetener;
f) Flavouring agent; and
g) one or more pharmaceutically acceptable excipient.

In yet another aspect of the present invention, there is provided a stable aqueous pharmaceutical composition of Topiramate comprising:
a) about 2.50% w/v of Topiramate;
b) about 48% w/v of Polyethylene glycol;
c) about 2.16% w/v of methyl paraben;
d) about 0.24% w/v of propyl paraben;
e) about 1% w/v of sucralose;
f) about 1.90% w/v of purified water;
g) about 1.9% w/v of propylene glycol;
h) about 0.05% w/v of mixed berry flavour; and
i) about 61.94 w/v of glycerin.

In one aspect of the present invention, there is provided a stable aqueous pharmaceutical composition of Topiramate comprising:
a) about 2.09% w/w of Topiramate;
b) about 40.17% w/w of Polyethylene glycol;
c) about 1.67% w/w of methyl paraben;
d) about 0.19% w/w of propyl paraben;
e) about 0.84% w/w of sucralose;
f) about 1.59% w/w of purified water;
g) about 1.59% w/w of propylene glycol;
h) about 0.05% w/w of mixed berry flavour; and
i) about 51.83% of glycerin.

It is yet another object of the present invention to provide a stable aqueous pharmaceutical composition of Topiramate comprising:
a) about 25 mg/ml of Topiramate;
b) about 480 mg/ml of Polyethylene glycol;
c) about 20 mg/ml of methyl paraben;
d) about 2.2 mg/ml of propyl paraben;
e) about 10 mg/ml of sucralose;
f) about 19 mg/ml of purified water;
g) about 19 mg/ml of propylene glycol;
h) about 0.45 mg/ml of mixed berry flavour; and
i) about 619.4 mg/ml of glycerin.

It is yet another object of the present invention to provide a stable aqueous pharmaceutical composition of Topiramate comprising:
a) about 10-30 mg/ml of Topiramate;
b) about 340-540 mg/ml of Polyethylene glycol;
c) about 12-25 mg/ml of methyl paraben;
d) about 1-5 mg/ml of propyl paraben;
e) about 4-16 mg/ml of sucralose;
f) about 10-30 mg/ml of purified water;
g) about 10-30 mg/ml of propylene glycol;
h) about 0.1-1 mg/ml of mixed berry flavour; and
i) about 530-670 mg/ml glycerin.

In another aspect of the present invention, there is provided a stable pharmaceutical composition of Topiramate comprising:
a) about 1-5% w/v of Topiramate;
b) about 25-65% w/v of Polyethylene glycol;
c) about 0.5-4.0% w/v of methyl paraben;
d) about 0.1-1% w/v of propyl paraben;
e) about 0.5-4% w/v of sucralose;
f) about 1-5% w/v of purified water;
g) about 1-5% w/v of propylene glycol;
h) about 0.01-2% w/v of mixed berry flavour; and
i) about 40-70% w/v of glycerin.

In yet another embodiment, solvent or solubilizing agents used in the composition of the present invention are selected from but are not limited to, ethanol, propanol, isopropanol, propylene glycol, polyethylene glycol of various molecular weights, dichloromethane, dimethylisosorbide, ethyl lactate, N-methylpyrrolidones, glycofurol, decaglycerol mono-, dioleate, triglycerol monooleate, polyglycerol oleate, mixed diesters of Caprylic/Capric acid and propylene glycol, ethyl oleate, glyceryl monooleate, Vitamin E TPGS, alpha tocopherol, or mixtures thereof. The preferred solvent or solubilizing agents is polyethylene glycol 400. The amount of solvent or solubilizing agents in the formulations of the present invention can range from, about 10% w/v to about 90% w/v; from about 15% w/v to about 65% w/v; from about 30% w/v to about 50% w/v, preferably about 48% w/v.

In another embodiment, the co-solvents used in the composition of the present invention are selected from but are not limited to, Glycerin, dimethylsulfoxide, N-methylpyrrolidone, dimethyl acetamide (DMA), dimethylsulfoxide (DMSO), dimethyl formamide, glycerol formal, ethoxy diglycol, triethylene glycol dimethyl ether, triacetin, diacetin, corn oil, acetyl triethyl citrate (ATC), ethyl lactate, polyglycolated capryl glycerides butyrolactone, dimethyl isosorbide, benzyl alcohol, ethanol, isopropyl alcohol, propylene glycol, purified water. The preferred co-solvent is Glycerin. The amount of co-solvents used in the formulations of the present invention can range from, about 50% w/v to about 70% w/v; from about 55% w/v to about 65% w/v; preferably about 63% w/v. The amount of Glycerin used in the aqueous formulations of the present invention is preferably about 62% w/v and the amount of Propylene glycol used in the aqueous formulations of the present invention is about 2% w/v.

In another embodiment, preservatives used in the composition of the present invention are selected from but are not limited to, benzyl benzoates, benzoic acid, benzyl alcohol, benzalkonium chloride, N-cetyl-N,N,N-trimethylammonium bromide (Cetrimide, Merck), chlorhexidine, chlorobutanol, chlorocresol, iminourea, parabens such as methyl-, ethyl-, propyl- or butylparaben, sodium methylparaben, sodium propylparaben, potassium sorbate, sodium benzoate, sodium propionate, phenol, phenoxyethanol, phenylethyl alcohol, phenylmercuric acetate, phenyl-mercuric borate, phenylmercuric nitrates, sorbic acid or thiomersal (sodium ethylmercurithiosalicylate), sepicide, tween 80, bithional, butyl p-hydroxy benzoate, p-chloro-m-xylenol, dehydro acetic acid, methyl-p-hydroxybenzoate, propyl-p-hydroxybenzoate and mixtures thereof. Methylparaben, propylparaben, sodium methylparaben and sodium propylparaben are the preferred preservatives. The amount of preservatives in the formulations of the present invention can range from, about 0.05% w/v to about 15% w/v, from about 1% w/v to 10% w/v, from about 1% w/v to 5% w/v, preferably about 2.22% w/v.

In another embodiment, the aqueous compositions of the present include a sweetener. As defined herein, the term “sweetener” is an excipient which provides sweetness to a pharmaceutical aqueous composition. Sweetener excipients may be selected from the group consisting of aspartame, mannitol, sorbitol and xylitol, dextrose, saccharin sodium, fructose, maltodextrin, sucralose, sucrose, and combinations thereof. The amount of sweetener that may present invention can range from about 4 mg/ml to about 16 mg/ml. Alternatively, the amount may range from about 0.5% w/v to about 4% w/v. Preferably, the sweetener used is Sucralose and is present in a concentration of about 1% w/v.

In another embodiment, the aqueous compositions of the present include a flavoring agent. The term “flavoring agent” or “Flavouring agent” means an excipient or a mixture of excipients or compounds which impart a pleasant flavor to the pharmaceutical composition. The flavoring agents are well known in the art and a person of ordinary skill in the art would easily identify the suitable flavoring agents to be used on pharmaceutical compositions. The amount of flavoring agent to be used may depend on a number of factors, including the organoleptic effect desired. The flavoring agent used in the compositions of the present invention is a mixed berry flavor and is present in a concentration of 0.01% w/v to about 2% w/v.

Further, the present invention provides a process for preparation of a stable aqueous pharmaceutical compositions comprising;
a) Topiramate, as an active ingredient;
b) Polyethylene glycol as solvent;
c) Co-solvent;
d) purified water;
e) Sweetener;
f) Flavouring agent; and
g) one or more pharmaceutically acceptable excipient;
   wherein the process comprises dissolving methyl paraben, propyl paraben and propylene glycol in polyethylene glycol 400 and adding Topiramate, sucralose, purified water, mixed berry flavor to obtain clear solution while and adjusting final volume using Glycerin.

An aspect of the present invention provides a stable aqueous pharmaceutical composition comprising topiramate for the treatment of epileptic disorders, migraine or disorders related to central nervous system.

The stable aqueous pharmaceutical compositions of the present invention may be packaged in a suitable package such a bottle.

It should be appreciated that the invention can be embodied/aspects in different forms and should not be construed as limited to the embodiments set forth herein. Rather, these embodiments are provided so that this disclosure will be thorough and complete, and will convey the scope of the invention to those skilled in the art. Other features and embodiments of the invention will become apparent from the following examples, which are given for illustration of the invention rather than for limiting its intended scope.

The following examples are intended to illustrate the scope of the present invention in all its aspects but not to limit it thereto.

### Example 1 A: Aqueous Oral Composition Comprising Topiramate

| S. No. | Name of Ingredients | Std. Qty. in mg/100 mL |
|---|---|---|
| 1 | Topiramate | 2.50 |
| 2 | Polyethylene glycol | 48.00 |
| 3 | Methyl paraben | 2.0 |
| 4 | Propyl paraben | 0.22 |
| 5 | Sucralose | 1.00 |
| 6 | Purified water | 1.90 |
| 7 | Propylene glycol | 1.90 |
| 8 | Mixed berry flavor | 0.05 |
| 9 | Glycerin | q.s. to 100 mL |

Qs: Quantity sufficient

### Example 1 B: Aqueous Oral Composition Comprising Topiramate

| S. No. | Name of Ingredients | % W/V |
|---|---|---|
| 1 | Topiramate | 1-5 |
| 2 | Polyethylene glycol | 25-65 |
| 3 | Methyl paraben | 0.5-4.0 |
| 4 | Propyl paraben | 0.1-1.0 |
| 5 | Sucralose | 0.5-4 |
| 6 | Purified water | 1-5 |
| 7 | Propylene glycol | 1-5 |
| 8 | Mixed berry flavor | 0.01-2 |
| 9 | Glycerin | q.s. to 100 mL |

Qs: Quantity sufficient

Manufacturing Process for Example 1A & 1B:
1. Methyl paraben, Propyl paraben and Propylene glycol in Polyethylene glycol 400 were dissolved together under stirring to get clear solution.
2. Topiramate active ingredient, sucralose, and purified water were added to step 1 under stirring to get a clear solution.
3. Mixed berry flavor was added in step 1 under continuous stirring.
4. Glycerin was added to volume make up to get the final volume of the solution.
5. Final Solution was filtered through 6μ or 10μ poly propylene filter to get final solution.
6. Final solution was filled in a white colour square shape HDPE bottle with CRC caps.

The formulations of the invention exemplified in Example 1A were evaluated for stability at conditions of 25° C.±2° C. and 60% RH±5% RH, 40° C.±2° C. and 75% RH±5% RH for initial and 3M and 6M (months).

### A. Stability Study: Example 1A at 25° C.±2° C. and 60% RH±5% RH for Initial, 3M and 6M (Months) in Upright Position

| Sr. | | | | | |
|---|---|---|---|---|---|
| No | Test | Specification | Initial | 3 Month | 6 Month |
| 1 | Assay (w/w, by HPLC) | NLT 90.0% and NMT | 101.2% | 102.3% | 103.7% |
| | A) Topiramate (25 mg/ml) | 110.0% of the labeled | | | |
| | | amount of Topiramate | | | |
| | | (C₁₂H₂₁NO₈S). | | | |
| | B) Methyl Paraben | NLT 50.0% and NMT | 102.5% | 102.2% | 102.8% |
| | (20 mg/ml) | 120.0% of the added | | | |
| | | amount of methyl | | | |
| | | paraben (C₈H₈O₃). | | | |
| | C) Propyl Paraben | NLT 50.0% and NMT | 102.7% | 101.7% | 103.5% |
| | (2.22 mg/ml) | 120.0% of the added | | | |
| | | amount of Propyl | | | |
| | | paraben (C₁₀H₁₂O₃). | | | |
| 2 | Water Content (w/w By | Between 1.30 and | 1.7% | 1.8% | 1.76% |
| | KF Titration) | 4.00% | | | |
| 3 | Organic Impurities | | | | |
| | (w/w, By HPLC) | | | | |
| | Topiramate Related | Not more than 0.5% | ND | ND | ND |
| | compound A | | | | |
| | Individual unspecified | Not more than 0.2% | ND | ND | ND |
| | degradation products | | | | |
| | Total degradation | Not more than 0.7% | ND | ND | ND |
| | products | | | | |
| 4 | Limit of 4-Hydroxy | Not more than 0.5% | 0.01% | BLQ | BLQ |
| | Benzoic acid | | | (0.008%) | (0.008%) |
| 5 | Microbial enumeration | | | | |
| | tests and Tests for | | | | |
| | specified | | | | |
| | microorganisms | | | | |
| | Total viable aerobic | Not more than 10² | <10 cfu/ml | NA | NA |
| | microbial count | cfu/ml | | | |
| | Total combined molds | Not more than 10¹ | <10 cfu/ml | | |
| | and yeast count | cfu/ml | | | |
| | Escherichia coli | Absent/1 ml | Absent | | |
| 6 | Limit of sulfate and | | | | |
| | sulfamate (By Metrohm | | | | |
| | ion chromatography | | | | |
| | Sulfate | Not more than 0.25% | ND | ND | BLQ |
| | | | | | (0.017%) |
| | Sulfamate | Not more than 0.25% | ND | BLQ | ND |
| | | | | (0.007%) | |

### B. Stability Study: Example 1A at 40° C.±2° C. and 75% RH±5% RH for Initial, 3M and 6M (Months) in Upright Position

| Sr. | | | | | |
|---|---|---|---|---|---|
| No | Test | Specification | Initial | 3 Month | 6 Month |
| 1 | Assay (w/w, by HPLC) | NLT 90.0% and NMT | 101.2% | 103.0% | 100.2% |
| | A) Topiramate (25 mg/ml) | 110.0% of the labeled | | | |
| | | amount of Topiramate | | | |
| | | (C₁₂H₂₁NO₈S). | | | |
| | B) Methyl Paraben | NLT 50.0% and NMT | 102.5% | 102.6% | 102.0% |
| | (20 mg/ml) | 120.0% of the added | | | |
| | | amount of methyl | | | |
| | | paraben (C₈H₈O₃). | | | |
| | C) Propyl Paraben | NLT 50.0% and NMT | 102.7% | 102.2% | 102.6% |
| | (2.22 mg/ml) | 120.0% of the added | | | |
| | | amount of Propyl | | | |
| | | paraben (C₁₀H₁₂O₃). | | | |
| 2 | Water Content (w/w By | Between 1.30 and | 1.7% | 1.8% | 1.91% |
| | KF Titration) | 4.00% | | | |
| 3 | Organic Impurities | | | | |
| | (w/w, By HPLC) | | | | |
| | Topiramate Related | Not more than 0.5% | ND | BLQ | 0.28% |
| | compound A | | | (0.153%) | |
| | Individual unspecified | Not more than 0.2% | ND | BLQ | BLQ |
| | degradation products | | | (0.101%) | (0.101%) |
| | Total degradation | Not more than 0.7% | ND | BLQ | 0.3% |
| | products | | | | |
| 4 | Limit of 4-Hydroxy | Not more than 0.5% | 0.01% | BLQ | 0.01% |
| | Benzoic acid | | | (0.008%) | |
| 5 | Microbial enumeration | | | | |
| | tests and Tests for | | | | |
| | specified | | | | |
| | microorganisms | | | | |
| | Total viable aerobic | Not more than 10² | <10 cfu/ml | NA | <10 cfu/ml |
| | microbial count | cfu/ml | | | |
| | Total combined molds | Not more than 10¹ | <10 cfu/ml | | <10 cfu/ml |
| | and yeast count | cfu/ml | | | |
| | Escherichia coli | Absent/1 ml | Absent | | Absent/1 ml |
| 6 | Limit of sulfate and | | | | |
| | sulfamate (By Metrohm | | | | |
| | ion chromatography | | | | |
| | Sulfate | Not more than 0.25% | ND | BLQ | BLQ |
| | | | | (0.017%) | (0.017%) |
| | Sulfamate | Not more than 0.25% | ND | BLQ | ND |
| | | | | (0.007%) | |

ND: Not Detected;
BLQ: Below Limit of Quantification;
NA: Not Applicable;
cfu: Colony forming unit

All the physical and chemical parameters were found satisfactory and the initial, 3M and 6M stability data at 25° C.±2° C. and 60% RH±5% RH, 40° C.±2° C. and 75% RH±5% RH was found to be satisfactory.

1. A stable aqueous solution composition suitable for oral administration comprising:
a) Topiramate, in a concentration of about 10 mg/ml to about 30 mg/ml;
b) a pharmaceutically acceptable solvent system consisting of Polyethylene glycol in a concentration of about 340 mg/ml to 540 mg/ml;
c) a pharmaceutically acceptable Co-solvent;
d) water;
e) sweetener;
f) One or more flavoring agent and is present in a concentration of about 0.1 mg/ml to about 1 mg/ml; and
g) a pharmaceutically acceptable excipient.

2. A stable aqueous solution composition as in claim 1, wherein the co-solvent selected is from the group consisting of glycerin, dimethylsulfoxide, N-methylpyrrolidone, dimethyl acetamide (DMA), dimethylsulfoxide (DMSO), dimethyl formamide, glycerol formal, ethoxy diglycol, triethylene glycol dimethyl ether, triacetin, diacetin, corn oil, acetyl triethyl citrate (ATC), ethyl lactate, polyglycolated capryl glycerides butyrolactone, dimethyl isosorbide, benzyl alcohol, ethanol, isopropyl alcohol and propylene glycol, and a combination thereof.

3. (canceled)

4. A stable aqueous solution composition as in claim 1, wherein the pharmaceutical excipient is a preservative selected from the group consisting of benzyl benzoates, benzoic acid, benzyl alcohol, benzalkonium chloride, parabens such as methyl-, ethyl-, propyl- or butylparaben, sodium methylparaben, sodium propylparaben, potassium sorbate, sodium benzoate, sodium propionate, phenol, phenoxyethanol, phenylethyl alcohol, tween 80, bithional, butyl p-hydroxy benzoate, p-chloro-m-xylenol, dehydro acetic acid, methyl-p-hydroxybenzoate, propyl-p-hydroxybenzoate and mixtures thereof.

5. (canceled)

6. A stable aqueous solution composition as in claim 1, wherein the water is present at a concentration of about 10 mg/ml to about 30 mg/ml.

7. A stable aqueous solution composition as in claim 1, wherein the sweetener is sucralose and is present in a concentration of about 4 mg/ml to about 16 mg/ml.

8. A stable aqueous solution composition suitable for oral administration consisting of:
a) Topiramate at a concentration of about 2.5% w/v;
b) Polyethylene glycol at concentration of about 25-65% w/v;
c) Methyl paraben at a concentration of about 0.5-4% w/v;
d) Propyl paraben at a concentration of about 0.1-1% w/v;
e) Sucralose at a concentration of about 0.5-4% w/v;
f) Purified water at a concentration of about 1-5% w/v;
g) Propylene glycol at a concentration of about 1-5% w/v;
h) Mixed berry flavour at a concentration of about 0.01-2% w/v; and
i) glycerin at a concentration of about 40-70% w/v.

9. A stable aqueous solution composition suitable for oral administration consisting of:
a) about 25 mg/ml of Topiramate;
b) about 480 mg/ml of Polyethylene glycol;
c) about 20 mg/ml of methyl paraben;
d) about 2.2 mg/ml of propyl paraben;
e) about 10 mg/ml of sucralose;
f) about 19 mg/ml of purified water;
g) about 19 mg/ml of propylene glycol;
h) about 0.45 mg/ml of mixed berry flavour; and
i) about 619.4 mg/ml of glycerin.

10. A stable aqueous solution composition suitable for oral administration consisting of:
a) about 15-30 mg/ml of Topiramate;
b) about 340-540 mg/ml of Polyethylene glycol;
c) about 12-25 mg/ml of methyl paraben;
d) about 1-5 mg/ml of propyl paraben;
e) about 4-16 mg/ml of sucralose;
f) about 10-30 mg/ml of purified water;
g) about 10-30 mg/ml of propylene glycol;
h) about 0.1-1 mg/ml of mixed berry flavour; and
i) about 530-670 mg/ml of glycerin.

11. A stable aqueous solution composition as in claim 4, wherein the co-solvent is glycerin and propylene glycol or combinations thereof present at a concentration of about 540 mg/ml to about 700 mg/ml.

12. A stable aqueous solution composition as in claim 6, wherein the preservative is methyl paraben and propyl paraben which are present at a concentration of about 12 mg/ml to about 30 mg/ml.
